(19) United States
(12) Patent Application Publication
(10) Pub. No.: US 2025/0099042 A1
(43) Pub. Date: Mar. 27, 2025

(54) VIBRATION DETECTION SYSTEM AND VIBRATION DETECTION METHOD

(71) Applicant: SONY SEMICONDUCTOR SOLUTIONS CORPORATION, KANAGAWA (JP)

(72) Inventors: OSAMU KOZAKAI, KANAGAWA (JP); BAHIEN CHU, KANAGAWA (JP)

(21) Appl. No.: 18/729,388

(22) PCT Filed: Jan. 12, 2023
(86) PCT No.: PCT/JP2023/000597

§ 371 Date: Jul. 16, 2024

(30) Foreign Application Priority Data
Jan. 25, 2022 (JP) 2022-009387

(51) Int. Cl. A61B 5/00; A61B 5/0205; A61B 5/11; A61B 5/113

(52) U.S. Cl. CPC A61B 5/6893; A61B 5/0205; A61B 5/1102; A61B 5/113

(57) ABSTRACT

A vibration detection system according to the present disclosure includes a vision sensor (55), a radar (56), and a control unit that controls the vision sensor (55) and the radar (56). Furthermore, the control unit includes a specifying unit (74) and an acquisition unit (75). The specifying unit (74) specifies a position of a vibration source in a measurement target by the vision sensor (55). The acquisition unit (75) acquires vibration information related to vibration at the specified position of the vibration source by the radar (56).

### FIELD

The present disclosure relates to a vibration detection system and a vibration detection method.

### BACKGROUND

In recent years, a sensor for detecting passengers in a vehicle compartment has been developed for the purpose of improving safety of cars and the like. This sensor is used for, for example, control of an airbag, control of automatic driving, and the like (see, for example, Patent Literature 1).

### CITATION LIST

### Patent Literature

Patent Literature 1: JP H8-127264 A

### SUMMARY

### Technical Problem

The present disclosure proposes a vibration detection system and a vibration detection method that can accurately detect micro vibrations.

### Solution to Problem

According to the present disclosure, there is provided a vibration detection system. The vibration detection system includes a vision sensor, a radar, and a control unit that controls the vision sensor and the radar. Furthermore, the control unit includes a specifying unit and an acquisition unit. The specifying unit specifies a position of a vibration source in a measurement target by the vision sensor. The acquisition unit acquires vibration information related to vibration at the specified position of the vibration source by the radar.

### BRIEF DESCRIPTION OF DRAWINGS

FIG. 1 is a block diagram illustrating a configuration example of a vehicle control system according to an embodiment of the present disclosure.

FIG. 2 is a diagram illustrating an example of a sensing area according to the embodiment of the present disclosure.

FIG. 3 is a block diagram illustrating a detailed configuration example of the vehicle control system according to the embodiment of the present disclosure.

FIG. 4 is a diagram illustrating an example of arrangement of a vision sensor and a radar according to the embodiment of the present disclosure.

FIG. 5 is a diagram for describing an example of processing executed by the vehicle control system according to the embodiment of the present disclosure.

FIG. 6 is a diagram for describing an example of the processing executed by the vehicle control system according to the embodiment of the present disclosure.

FIG. 7 is a diagram for describing an example of the processing executed by the vehicle control system according to modification 1 of the embodiment of the present disclosure.

FIG. 8 is a diagram for describing an example of the processing executed by the vehicle control system according to modification 1 of the embodiment of the present disclosure.

FIG. 9 is a diagram for describing an example of the processing executed by the vehicle control system according to modification 2 of the embodiment of the present disclosure.

FIG. 10 is a diagram for describing an example of the processing executed by the vehicle control system according to modification 3 of the embodiment of the present disclosure.

FIG. 11 is a diagram for describing an example of the processing executed by the vehicle control system according to modification 4 of the embodiment of the present disclosure.

FIG. 12 is a diagram for describing an example of the processing executed by the vehicle control system according to modification 4 of the embodiment of the present disclosure.

FIG. 13 is a flowchart illustrating an example of a procedure of detection processing executed by the vehicle control system according to the embodiment of the present disclosure.

FIG. 14 is a flowchart illustrating an example of the procedure of the detection processing executed by the vehicle control system according to modification 1 of the embodiment of the present disclosure.

FIG. 15 is a flowchart illustrating an example of the procedure of the detection processing executed by the vehicle control system according to modification 2 of the embodiment of the present disclosure.

FIG. 16 is a flowchart illustrating an example of the procedure of the detection processing executed by the vehicle control system according to modification 3 of the embodiment of the present disclosure.

### DESCRIPTION OF EMBODIMENTS

Hereinafter, an embodiment of the present disclosure will be described with reference to the accompanying drawings. Note that the present disclosure is not limited to the following embodiment. Furthermore, each embodiment can be combined as appropriate within a range without making processing contents contradict each other. Furthermore, the same parts will be assigned the same reference numerals in the following embodiment, and redundant description will be omitted.

In recent years, a sensor for detecting passengers in a vehicle compartment has been developed for the purpose of improving safety of cars and the like. This sensor is used for, for example, control of an airbag, control of automatic driving, and the like.

However, the above-described conventional technique can detect the presence/absence of passengers in the vehicle compartment, yet has great difficulty in accurately detecting vital information (e.g., a heart rate, a respiratory rate, and the like) of the passenger.

Hence, it is expected to implement a technology that can overcome the above-described problem and accurately detect micro vibrations caused by heartbeat, respiration, or the like of the passenger.

### Configuration Example of Vehicle Control System

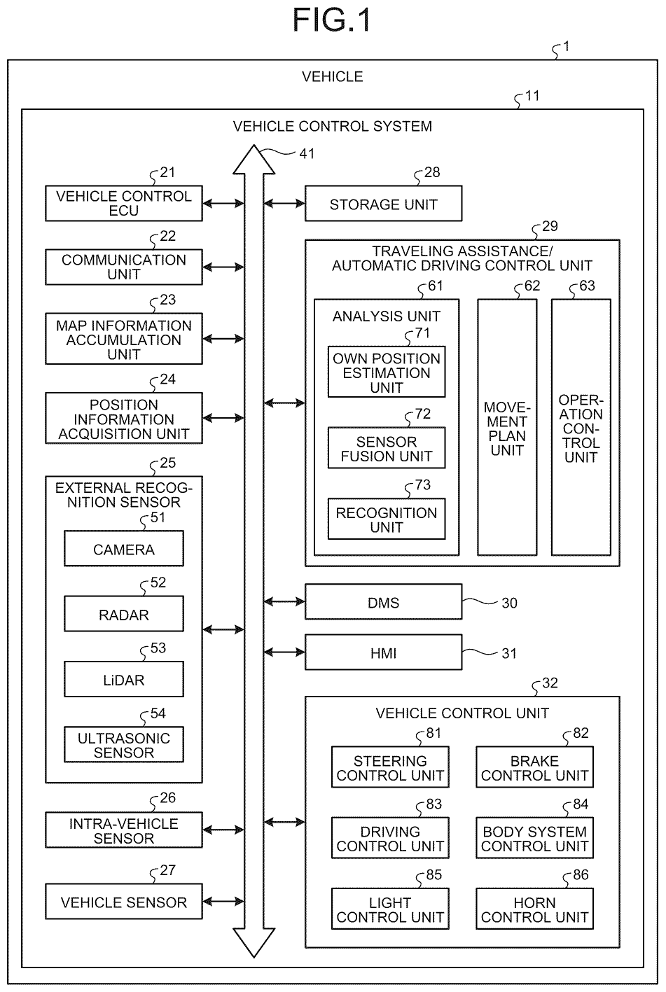

FIG. 1 is a block diagram illustrating a configuration example of a vehicle control system 11 that is an example of a mobile device control system to which the technique according to the present technique is applicable. The vehicle control system 11 is an example of a vibration detection system.

The vehicle control system 11 is provided in a vehicle 1, and performs processing related to traveling assistance and automatic driving of the vehicle 1.

The vehicle control system 11 includes a vehicle control Electronic Control Unit (ECU) 21, a communication unit 22, a map information accumulation unit 23, a position information acquisition unit 24, an external recognition sensor 25, an intra-vehicle sensor 26, a vehicle sensor 27, a storage unit 28, a traveling assistance/automatic driving control unit 29, a Driver Monitoring System (DMS) 30, a Human Machine Interface (HMI) 31, and a vehicle control unit 32. The traveling assistance/automatic driving control unit 29 is an example of a control unit.

The vehicle control ECU 21, the communication unit 22, the map information accumulation unit 23, the position information acquisition unit 24, the external recognition sensor 25, the intra-vehicle sensor 26, the vehicle sensor 27, the storage unit 28, the traveling assistance/automatic driving control unit 29, the Driver Monitoring System (DMS) 30, the Human Machine Interface (HMI) 31, and the vehicle control unit 32 are communicably connected to each other via a communication network 41.

The communication network 41 is configured as, for example, an in-vehicle communication network that conforms to a digital bidirectional communication standards such as a Controller Area Network (CAN), a Local Interconnect Network (LIN), a Local Area Network (LAN), FlexRay (registered trademark), or the Ethernet (registered trademark), a bus, or the like. The communication network 41 may be selectively used according to the type of data to be transmitted. For example, the CAN may be applied to data related to vehicle control, and the Ethernet may be applied to large-volume data. Note that there is also a case where each unit of the vehicle control system 11 is directly connected using, for example, wireless communication that assumes communication at a relatively short distance, such as Near Field Communication (NFC) or Bluetooth (registered trademark) without the communication network 41.

Note that, in a case where each unit of the vehicle control system 11 performs communication via the communication network 41, description of the communication network 41 will be omitted hereinafter. A case where, for example, the vehicle control ECU 21 and the communication unit 22 communicate via the communication network 41 will be simply described to read that the vehicle control ECU 21 and the communication unit 22 communicate.

The vehicle control ECU 21 includes, for example, various processors such as a Central Processing Unit (CPU) and a Micro Processing Unit (MPU). The vehicle control ECU 21 controls all or part of the functions of the vehicle control system 11.

The communication unit 22 communicates with various devices inside and outside the vehicle, other vehicles, servers, base stations, and the like, and transmits and receives various data. At this time, the communication unit 22 can perform communication using a plurality of communication methods.

Communication that can be executed by the communication unit 22 with the outside of the vehicle will be schematically described. The communication unit 22 can communicate with a server (hereinafter, referred to as an external server) or the like existing on an external network via a base station or an access point by a wireless communication method such as the 5th generation mobile communication system (5G), Long Term Evolution (LTE), or Dedicated Short Range Communications (DSRC). The external network that the communication unit 22 communicates with is, for example, the Internet, a cloud network, a network unique to a business operator, or the like. The communication method performed by the communication unit 22 with the external network is not particularly limited as long as the communication method is a wireless communication method that can perform digital bidirectional communication at a predetermined communication speed or more and at a predetermined distance equal or more.

Furthermore, for example, the communication unit 22 can communicate with a terminal that exists near an own vehicle using a Peer to Peer (P2P) technique. Examples of the terminal existing near the own vehicle include a terminal that is equipped by a moving body such as a pedestrian or a bicycle moving at a relatively low speed, a terminal that is installed in a store or the like with a position fixed, and a Machine Type Communication (MTC) terminal. Furthermore, the communication unit 22 can also perform V2X communication. The V2X communication refers to, for example, communication between the own vehicle and another vehicle or device such as vehicle to vehicle communication with another vehicle, vehicle to infrastructure communication with a roadside device or the like, vehicle to home communication, and vehicle to pedestrian communication with a terminal or the like owned by a pedestrian, and the like.

The communication unit 22 can receive from the outside, for example, a program for updating software for controlling the operation of the vehicle control system 11 (Over The Air). Furthermore, the communication unit 22 can receive map information, traffic information, information of surroundings of the vehicle 1, and the like from the outside. Furthermore, for example, the communication unit 22 can transmit information related to the vehicle 1, information of the surroundings of the vehicle 1, and the like to the outside. Examples of the information related to the vehicle 1 and transmitted to the outside by the communication unit 22 include data indicating the state of the vehicle 1, a recognition result of the recognition unit 73, and the like. Furthermore, for example, the communication unit 22 performs communication that supports a vehicle emergency call system such as an eCall.

For example, the communication unit 22 receives an electromagnetic wave transmitted by a Vehicle Information and Communication System (VICS) (registered trademark) such as a radio wave beacon, an optical beacon, or FM multiplex broadcasting.

Furthermore, communication that can be executed by the communication unit 22 with a vehicle interior will be schematically described. The communication unit 22 can communicate with each device in the vehicle using, for example, wireless communication. The communication unit 22 can perform wireless communication with an in-vehicle device by a communication method that can perform digital bidirectional communication at a predetermined communication speed or more by wireless communication such as a wireless LAN, Bluetooth, NFC, or a wireless USB (WUSB). The communication unit 22 is not limited thereto, and can also communicate with each device in the vehicle using wired communication. For example, the communication unit 22 can communicate with each device in the vehicle by wired communication via a cable connected to an unillustrated connection terminal. The communication unit 22 can communicate with each device in the vehicle by a communication method that can perform digital bidirectional communication at a predetermined communication speed or more by wired communication such as a Universal Serial Bus (USB), a High-Definition Multimedia Interface (HDMI) (registered trademark), or a Mobile High-definition Link (MHL).

Here, the intra-vehicle device refers to, for example, a device that is not connected to the communication network 41 in the vehicle. Examples of the intra-vehicle device are assumed as mobile devices and wearable devices that are owned by passengers such as a driver, information devices that are brought into the vehicle and temporarily installed, and the like.

The map information accumulation unit 23 accumulates one or both of a map acquired from the outside and a map created by the vehicle 1. For example, the map information accumulation unit 23 accumulates a three-dimensional highly accurate map, a global map that has lower accuracy and covers a wider area than the highly accurate map, and the like.

The highly accurate map is, for example, a dynamic map, a point cloud map, a vector map, or the like. The dynamic map is, for example, a map that includes four layers of dynamic information, semi-dynamic information, semi-static information, and static information, and is provided to the vehicle 1 from an external server or the like. The point cloud map is a map that includes point clouds (point cloud data). The vector map is, for example, a map that is obtained by associating, for example, traffic information such as lanes and positions of traffic lights with a point cloud map, and is adapted to an Advanced Driver Assistance System (ADAS) or Autonomous driving (AD).

The point cloud map and the vector map may be provided from, for example, an external server or the like, or may be created by the vehicle 1 as maps to be matched with a local map to be described later based on sensing results of a camera 51, a radar 52, a LiDAR 53, or the like, and accumulated in the map information accumulation unit 23. Furthermore, in a case where the highly accurate map is provided from an external server or the like, for example, map data of several hundred square meters related to plan paths on which the vehicle 1 travels from now is acquired from the external server or the like to reduce a communications traffic.

The position information acquisition unit 24 receives a Global Navigation Satellite System (GNSS) signal from a GNSS satellite, and acquires position information of the vehicle 1. The acquired position information is supplied to the traveling assistance/automatic driving control unit 29. Note that the position information acquisition unit 24 is not limited to a method that uses the GNSS signal, and may acquire the position information using, for example, a beacon.

The external recognition sensor 25 includes various sensors that are used for recognizing a situation outside the vehicle 1, and supplies sensor data from each sensor to each unit of the vehicle control system 11. The type and the number of sensors included in the external recognition sensor 25 are arbitrary.

For example, the external recognition sensor 25 includes the camera 51, the radar 52, the Light Detection and Ranging or the Laser imaging Detection and Ranging (LiDAR) 53, and an ultrasonic sensor 54. The external recognition sensor 25 is not limited thereto, and may employ a configuration including one or more types of sensors among the camera 51, the radar 52, the LiDAR 53, and the ultrasonic sensor 54. The numbers of the cameras 51, the radars 52, the LiDARs 53, and the ultrasonic sensors 54 are not particularly limited as long as the cameras 51, the radars 52, the LiDARs 53, and the ultrasonic sensors 54 can be practically installed in the vehicle 1. Furthermore, the type of a sensor included in the external recognition sensor 25 is not limited to this example, and the external recognition sensor 25 may include other types of sensors. An example of a sensing area of each sensor included in the external recognition sensor 25 will be described later.

Note that a photographing method of the camera 51 is not particularly limited. For example, cameras of various photographing methods such as a Time-of-Flight (ToF) camera, a stereo camera, a monocular camera, and an infrared camera that adopt photographing methods that can measure distances are applicable to the camera 51 as necessary. The camera 51 is not limited thereto, and may simply acquire a captured image regardless of distance measurement.

Furthermore, for example, the external recognition sensor 25 can include an environment sensor for detecting an environment for the vehicle 1. The environment sensor is a sensor for detecting an environment such as a weather, an atmospheric phenomena, and brightness, and can include various sensors such as a raindrop sensor, a fog sensor, a sunshine sensor, a snow sensor, and an illuminance sensor.

Furthermore, for example, the external recognition sensor 25 includes a microphone used for, for example, detecting a sound around the vehicle 1, a position of a sound source, and the like.

The intra-vehicle sensor 26 includes various sensors for detecting information of the vehicle interior, and supplies sensor data from each sensor to each unit of the vehicle control system 11. Types and the numbers of various sensors included in the intra-vehicle sensor 26 are not particularly limited as long as the types and the numbers of various sensors are types and the numbers that can be practically installed in the vehicle 1.

For example, the intra-vehicle sensor 26 can include one or more sensors of a camera, a radar, a seating sensor, a steering wheel sensor, a microphone, and a biosensor. As the camera included in the intra-vehicle sensor 26, for example, cameras of various photographing methods that can perform distance measurement, such as a ToF camera, a stereo camera, a monocular camera, and an infrared camera, can be used. The camera included in the intra-vehicle sensor 26 is not limited thereto, and may simply acquire a captured image regardless of distance measurement. The biosensor included in the intra-vehicle sensor 26 is provided to, for example, a seat, a steering wheel, or the like, and detects various pieces of biological information of passengers such as a driver. Details of the intra-vehicle sensor 26 will be described later.

The vehicle sensor 27 includes various sensors for detecting the state of the vehicle 1, and supplies sensor data from each sensor to each unit of the vehicle control system 11. Types and the numbers of various sensors included in the vehicle sensor 27 are not particularly limited as long as the types and the numbers of various sensors are types and the numbers that can be practically installed in the vehicle 1.

For example, the vehicle sensor 27 can include a speed sensor, an acceleration sensor, an angular velocity sensor (gyro sensor), and an Inertial Measurement Unit (IMU) that is obtained by integrating these sensors. For example, the vehicle sensor 27 includes a steering angle sensor that detects a steering angle of a steering wheel, a yaw rate sensor, an accelerator sensor that detects an operation amount of an accelerator pedal, and a brake sensor that detects an operation amount of a brake pedal. For example, the vehicle sensor 27 includes a rotation sensor that detects rotational speeds of an engine and a motor, an air pressure sensor that detects air pressures of tires, a slip ratio sensor that detects the slip ratio of the tire, and a wheel speed sensor that detects rotation speeds of wheels. For example, the vehicle sensor 27 includes a battery sensor that detects a remaining amount and a temperature of a battery, and an impact sensor that detects an impact from the outside.

The storage unit 28 includes at least one of a nonvolatile storage medium and a volatile storage medium, and stores data and programs. The storage unit 28 is used as, for example, an Electrically Erasable Programmable Read Only Memory (EEPROM) and a Random Access Memory (RAM), and a magnetic storage device such as a Hard Disc Drive (HDD), a semiconductor storage device, an optical storage device, and a magneto-optical storage device is applicable as the storage medium. The storage unit 28 stores various programs and data used by each unit of the vehicle control system 11. For example, the storage unit 28 includes an Event Data Recorder (EDR) and a Data Storage System for Automated Driving (DSSAD), and stores information of the vehicle 1 before and after events such as an accident, and information acquired by the intra-vehicle sensor 26.

The traveling assistance/automatic driving control unit 29 controls traveling assistance and automatic driving of the vehicle 1. For example, the traveling assistance/automatic driving control unit 29 includes an analysis unit 61, a movement plan unit 62, and an operation control unit 63.

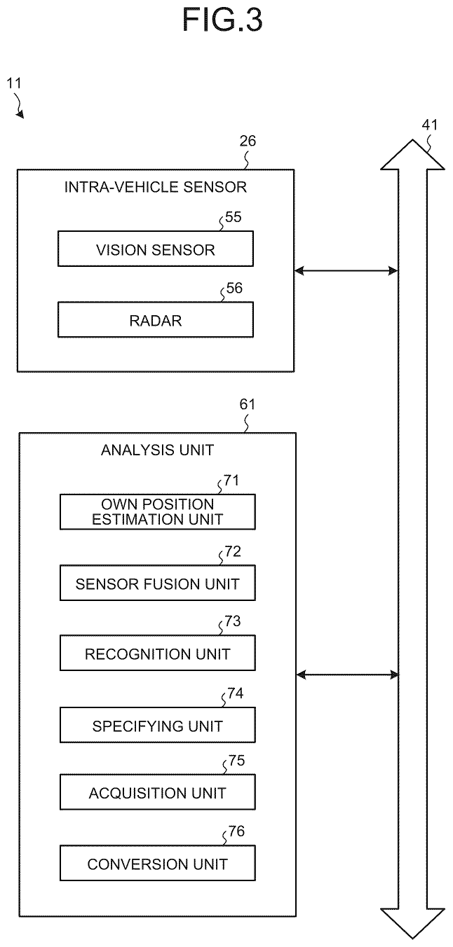

The analysis unit 61 performs analysis processing on the situation of the vehicle 1 and the surroundings. The analysis unit 61 includes an own position estimation unit 71, a sensor fusion unit 72, and a recognition unit 73. Furthermore, the analysis unit 61 according to the embodiment further includes a specifying unit 74 (see FIG. 3), an acquisition unit 75 (see FIG. 3), and a conversion unit 76 (see FIG. 3).

The own position estimation unit 71 estimates an own position of the vehicle 1 based on the sensor data from the external recognition sensor 25 and the highly accurate map accumulated in the map information accumulation unit 23. For example, the own position estimation unit 71 generates a local map based on the sensor data from the external recognition sensor 25, and estimates the own position of the vehicle 1 by matching the local map with the highly accurate map. The position of the vehicle 1 is based on, for example, the center of the rear wheel pair axle.

The local map is, for example, a three-dimensional highly accurate map that is created using a technique such as Simultaneous Localization and Mapping (SLAM), an occupancy grid map, or the like. The three-dimensional highly accurate map is, for example, the above-described point cloud map or the like. The occupancy grid map is a map in which a three-dimensional or two-dimensional space of the surroundings of the vehicle 1 is divided into grids (lattices) of a predetermined size, and that indicates an occupancy state of an object in units of grids. The occupancy state of the object is indicated by, for example, the presence/absence or an existence probability of the object. The local map is also used by the recognition unit 73 to perform, for example, detection processing and recognition processing on a situation outside the vehicle 1.

Note that the own position estimation unit 71 may estimate the own position of the vehicle 1 based on the position information acquired by the position information acquisition unit 24 and the sensor data from the vehicle sensor 27.

The sensor fusion unit 72 performs sensor fusion processing of combining a plurality of different types of sensor data (e.g., image data supplied from the camera 51 and sensor data supplied from the radar 52), and obtaining new information. Methods for combining different types of sensor data include integration, fusion, federation, and the like.

The recognition unit 73 executes detection processing of detecting a situation outside the vehicle 1, and recognition processing of recognizing the situation outside the vehicle 1.

For example, the recognition unit 73 performs the detection processing and the recognition processing on the situation outside the vehicle 1 based on information from the external recognition sensor 25, information from the own position estimation unit 71, information from the sensor fusion unit 72, and the like.

More specifically, for example, the recognition unit 73 performs detection processing, recognition processing, and the like on objects around the vehicle 1. The object detection processing refers to, for example, processing of detecting presence/absence, sizes, shapes, positions, motions, and the like of objects. The object recognition processing refers to, for example, processing of recognizing an attribute such as a type of an object or identifying a specific object. In this regard, the detection processing and the recognition processing are not necessarily clearly separated, and may overlap.

For example, the recognition unit 73 detects objects around the vehicle 1 by performing clustering for classifying point clouds that are based on the sensor data of the radar 52, the LiDAR 53, or the like into clusters of point clouds. As a result, the presence/absence, the sizes, the shapes, and the positions of the objects around the vehicle 1 are detected.

For example, the recognition unit 73 detects motions of the objects around the vehicle 1 by performing tracking for tracking the motions of the clusters of the point clouds classified by clustering. As a result, speeds and traveling directions (movement vectors) of the objects around the vehicle 1 are detected.

For example, the recognition unit 73 detects or recognizes vehicles, people, bicycles, obstacles, structures, roads, traffic lights, traffic signs, road signs, and the like based on the image data supplied from the camera 51. Furthermore, the recognition unit 73 may recognize types of the objects around the vehicle 1 by performing recognition processing such as semantic segmentation.

For example, the recognition unit 73 can perform recognition processing on traffic rules around the vehicle 1 based on the maps accumulated in the map information accumulation unit 23, the estimation result of the own position obtained by the own position estimation unit 71, and a recognition result of the objects around the vehicle 1 obtained by the recognition unit 73. This processing enables the recognition unit 73 to recognize positions and states of the traffic lights, contents of the traffic signs and the road signs, the contents of the traffic regulation, travelable lanes, and the like.

For example, the recognition unit 73 can perform recognition processing on the environment around the vehicle 1. As the recognition target environment of the surroundings for the recognition unit 73, the weather, the temperature, humidity, brightness, states of road surfaces, and the like are assumed.

Details of the analysis unit 61 according to the embodiment including the specifying unit 74, the acquisition unit 75, and the conversion unit 76 that are not illustrated in FIG. 1 will be described later.

The movement plan unit 62 creates a movement plan of the vehicle 1. For example, the movement plan unit 62 creates a movement plan by performing processing of global path planning and path tracking.

Note that the global path planning is processing of planning a rough path from a start to a goal. This global path planning is called trajectory planning, and also includes processing of performing local path planning that enables safe and smooth traveling near the vehicle 1 taking mobility characteristics of the vehicle 1 into account in the planned path.

Path tracking is processing of planning an operation for safely and accurately traveling a path planned by global path planning within a planned time. The movement plan unit 62 can calculate a target speed and a target angular velocity of the vehicle 1 based on, for example, a result of this path tracking processing.

The operation control unit 63 controls the operation of the vehicle 1 to achieve the movement plan created by the movement plan unit 62.

For example, the operation control unit 63 controls a steering control unit 81, a brake control unit 82, and a driving control unit 83 included in the vehicle control unit 32 to be described later, and performs acceleration/deceleration control and direction control such that the vehicle 1 travels on a trajectory calculated by trajectory planning. For example, the operation control unit 63 performs cooperative control for the purpose of implementing the functions of the ADAS such as collision avoidance or impact mitigation, tracking traveling, vehicle speed maintaining traveling, collision warning of an own vehicle, lane deviation warning of the own vehicle, and the like. For example, the operation control unit 63 performs cooperative control for the purpose of automatic driving or the like for performing autonomous traveling without depending on a driver's operation.

The DMS 30 performs processing of authenticating the driver, processing of recognizing a driver's state, and the like based on the sensor data from the intra-vehicle sensor 26, input data input to the HMI 31 described later, and the like. As the state of the recognition target driver, for example, a physical condition, a wakefulness level, a concentration level, a fatigue level, a line-of-sight direction, a drunkenness level, a driving operation, a posture, and the like are assumed.

Note that the DMS 30 may perform processing of authenticating a passenger other than the driver, and processing of recognizing the state of the passenger. Furthermore, for example, the DMS 30 may perform processing of recognizing the situation in the vehicle based on the sensor data from the intra-vehicle sensor 26. As the recognition target situation in the vehicle, for example, the temperature, humidity, brightness, smell, and the like are assumed.

The HMI 31 inputs various data, instructions, and the like, and presents various data to the driver and the like.

Data input by the HMI 31 will be schematically described. The HMI 31 includes an input device for a person to input data. The HMI 31 generates an input signal based on the data, the instruction, or the like input by the input device, and supplies the input signal to each unit of the vehicle control system 11. The HMI 31 includes operators such as a touch panel, a button, a switch, and a lever as the input device. The HMI 31 is not limited thereto, and may further include an input device that can input information by methods such as a voice, a gesture, and the like other than a manual operation. Furthermore, the HMI 31 may use, for example, a remote control device that uses infrared rays or radio waves, or an external connection device such as a mobile device or a wearable device that supports the operation of the vehicle control system 11 as an input device.

Presentation of data by the HMI 31 will be schematically described. The HMI 31 generates visual information, auditory information, and tactile information for passengers or the outside of the vehicle. Furthermore, the HMI 31 performs output control for controlling output, output contents, an output timing, an output method, and the like of each generated information. The HMI 31 generates and outputs information indicated by images or light such as an operation screen, a state indication of the vehicle 1, a warning indication, a monitor image indicating a situation around the vehicle 1, and the like as the visual information. Furthermore, the HMI 31 generates and outputs information indicated by sounds such as voice guidance, a warning sound, and a warning message, and the like as the auditory information. Furthermore, the HMI 31 generates and outputs information given to the tactile sense of the passenger by, for example, a force, vibration, a motion, or the like as the tactile information.

As an output device through which the HMI 31 outputs visual information, for example, a display device that presents visual information by displaying an image by itself or a projector device that presents visual information by projecting an image is applicable. Note that, in addition to a display device having a normal display, the display device may be a device that displays visual information in the field of view of the passenger, such as a head-up display, a transmission-type display, or a wearable device having an Augmented Reality (AR) function. Furthermore, the HMI 31 can also use display devices included in a navigation device, an instrument panel, a Camera Monitoring System (CMS), an electronic mirror, a lamp, or the like provided in the vehicle 1 as output devices that output visual information.

As the output device from which the HMI 31 outputs the auditory information, for example, an audio speaker, a headphone, or an earphone is applicable.

As an output device from which the HMI 31 outputs tactile information, for example, a haptic element that uses a haptic technique is applicable. The haptic element is provided at, for example, a portion such as a steering wheel or a seat that the passenger of the vehicle 1 contacts.

The vehicle control unit 32 controls each unit of the vehicle 1. The vehicle control unit 32 includes the steering control unit 81, the brake control unit 82, the driving control unit 83, a body system control unit 84, a light control unit 85, and a horn control unit 86.

For example, the steering control unit 81 detects and controls a state of a steering system of the vehicle 1. The steering system includes, for example, a steering mechanism that includes steering wheels and the like, an electric power steering, and the like. The steering control unit 81 includes, for example, a steering ECU that controls the steering system, an actuator that drives the steering system, and the like.

For example, the brake control unit 82 detects and controls a state of a brake system of the vehicle 1. The brake system includes, for example, a brake mechanism that includes a brake pedal and the like, an Antilock Brake System (ABS), a regenerative brake mechanism, and the like. The brake control unit 82 includes, for example, a brake ECU that controls the brake system, an actuator that drives the brake system, and the like.

For example, the driving control unit 83 detects and controls a state of a driving system of the vehicle 1. The driving system includes, for example, a driving force generation device for generating a driving force of an accelerator pedal, an internal combustion engine, a driving motor, or the like, a driving force transmission mechanism for transmitting the driving force to wheels, and the like. The driving control unit 83 includes, for example, a driving ECU that controls the driving system, an actuator that drives the driving system, and the like.

For example, the body system control unit 84 detects and controls a state of a body system of the vehicle 1. The body system includes, for example, a keyless entry system, a smart key system, a power window device, power seats, an air conditioner, airbags, seat belts, a shift lever, and the like. The body system control unit 84 includes, for example, a body system ECU that controls the body system, an actuator that drives the body system, and the like.

For example, the light control unit 85 detects and controls states of various lights of the vehicle 1. As the control target light, for example, a headlight, a backlight, a fog light, a turn signal, a brake light, a projection light, an indicator of a bumper, and the like are assumed. The light control unit 85 includes a light ECU that controls the light, an actuator that drives the light, and the like.

For example, the horn control unit 86 detects and controls a state of a car horn of the vehicle 1. The horn control unit 86 includes, for example, a horn ECU that controls the car horn, an actuator that drives the car horn, and the like.

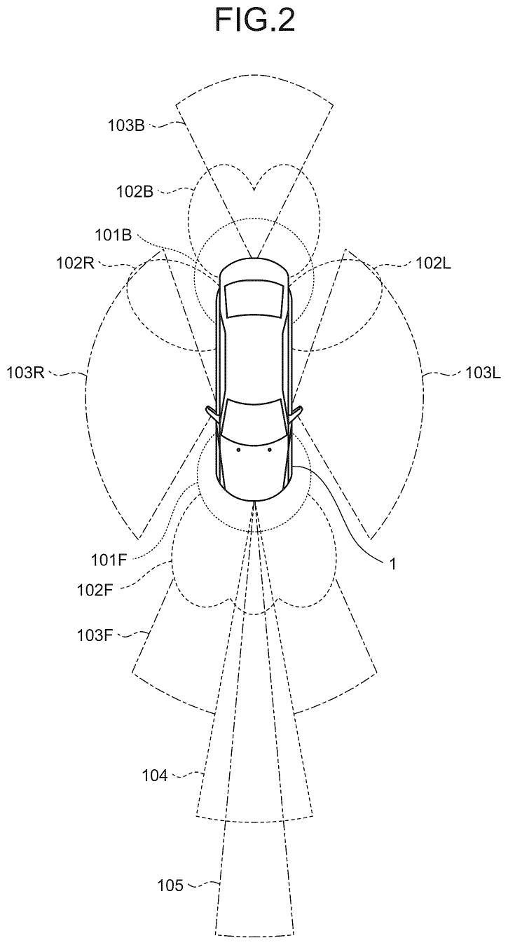

FIG. 2 is a view illustrating an example of sensing areas of the camera 51, the radar 52, the LiDAR 53, the ultrasonic sensor 54, and the like of the external recognition sensor 25 in FIG. 1. Note that FIG. 2 schematically illustrates the vehicle 1 from above, and the left end side is the front end (front) side of the vehicle 1 and the right end side is the rear end (rear) side of the vehicle 1.

A sensing area 101F and a sensing area 101B indicate examples of the sensing areas of the ultrasonic sensor 54. The sensing area 101F covers the periphery of the front end of the vehicle 1 by the plurality of ultrasonic sensors 54. The sensing area 101B covers the periphery of the rear end of the vehicle 1 by the plurality of ultrasonic sensors 54.

The sensing results in the sensing area 101F and the sensing area 101B are used to, for example, assist parking of the vehicle 1.

A sensing area 102F or a sensing area 102B indicates an example of a sensing area of the radar 52 for a short distance or a middle distance. The sensing area 102F covers a position farther than the sensing area 101F in front of the vehicle 1. The sensing area 102B covers a position farther than the sensing area 101B at the back of the vehicle 1. A sensing area 102L covers surroundings at the back of the left side surface of the vehicle 1. A sensing area 102R covers surroundings at the back of the right side surface of the vehicle 1.

A sensing result in the sensing area 102F is used to, for example, detect a vehicle, a pedestrian, or the like existing in front of the vehicle 1. A sensing result in the sensing area 102B is used for, for example, a collision prevention function at the back of the vehicle 1 or the like. Sensing results in the sensing area 102L and the sensing area 102R are used to, for example, detect an object in a blind spot at the side of the vehicle 1.

A sensing area 103F to a sensing area 103B indicate examples of sensing areas by the camera 51. The sensing area 103F covers a position farther than the sensing area 102F in front of the vehicle 1. The sensing area 103B covers a position farther than the sensing area 102B at the back of the vehicle 1. A sensing area 103L covers surroundings of the left side surface of the vehicle 1. A sensing area 103R covers surroundings of the right side surface of the vehicle 1.

A sensing result in the sensing area 103F can be used for, for example, recognition of traffic lights or traffic signs, a lane deviation prevention assist system, and an automatic headlight control system. A sensing result in the sensing area 103B can be used for, for example, parking assistance and a surround view system. Sensing results in the sensing area 103L and the sensing area 103R can be used for the surround view system, for example.

A sensing area 104 indicates an example of a sensing area of the LiDAR 53. The sensing area 104 covers a position farther than the sensing area 103F in front of the vehicle 1. On the other hand, the sensing area 104 has a narrower range in the left/right direction than the sensing area 103F.

A sensing result in the sensing area 104 is used to, for example, detect objects such as surrounding vehicles.

A sensing area 105 indicates an example of the sensing area of the long-range radar 52. The sensing area 105 covers a position farther than the sensing area 104 in front of the vehicle 1. On the other hand, the sensing area 105 has a narrower range in the left/right direction than the sensing area 104.

A sensing result in the sensing area 105 is used for, for example, Adaptive Cruise Control (ACC), emergency brake, collision avoidance, and the like.

Note that the sensing areas of the sensors of the camera 51, the radar 52, the LiDAR 53, and the ultrasonic sensor 54 included in the external recognition sensor 25 may employ various configurations other than those in FIG. 2. More specifically, the ultrasonic sensor 54 may also sense the sides of the vehicle 1, or the LiDAR 53 may sense the rear side of the vehicle 1. Furthermore, the installation position of each sensor is not limited to each of the above-described examples. Furthermore, the number of each sensor may be one or plural.

### Details of Control Processing

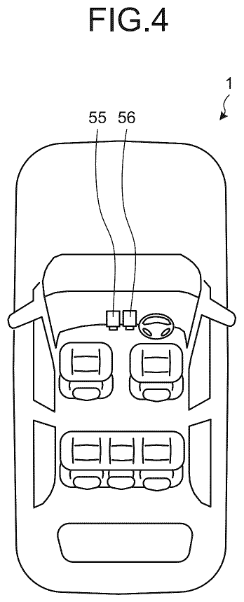

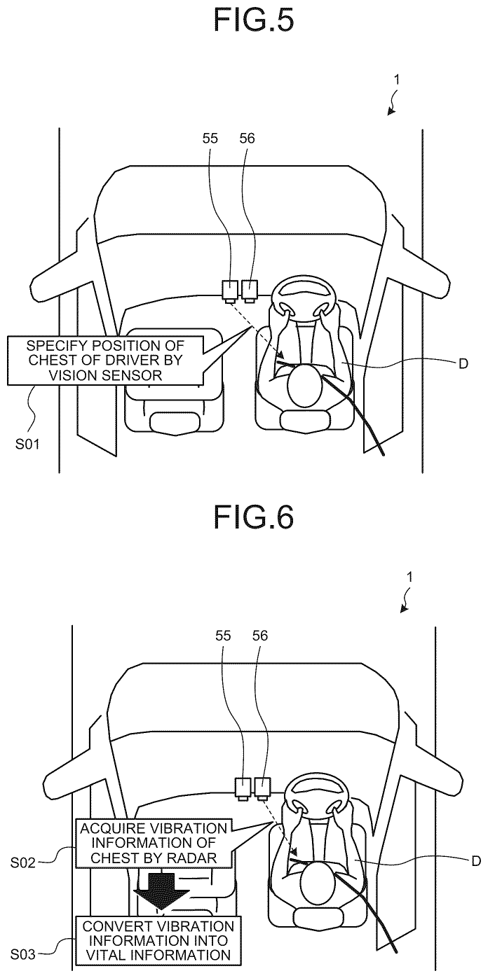

Next, details of the control processing according to the embodiment will be described with reference to FIGS. 3 to 6. FIG. 3 is a block diagram illustrating a detailed configuration example of the vehicle control system 11 according to the embodiment of the present disclosure, and FIG. 4 is a diagram illustrating an example of arrangement of a vision sensor 55 and a radar 56 according to the embodiment of the present disclosure. Furthermore, FIGS. 5 and 6 are diagrams for describing an example of processing executed by the vehicle control system 11 according to the embodiment of the present disclosure.

As illustrated in FIG. 3, the intra-vehicle sensor 26 according to the embodiment includes the vision sensor 55 and the radar 56. The vision sensor 55 is a sensor that can image a situation in the observation area, and is, for example, an RGB camera, an InRrared (IR) camera, a Time of Flight (ToF) sensor, an Event-based Vision Sensor (EVS), or the like.

The radar 56 transmits a radio wave of a predetermined band (e.g., millimeter waves or the like), receives a radio wave reflected from an object in the observation area, and thereby measures a distance to the object, a direction of the object, a speed of the object, and the like.

As illustrated in FIG. 4, the vision sensor 55 and the radar 56 are disposed in front of the vehicle interior of the vehicle 1 (near, for example, the overhead console or the like), and are installed such that a predetermined area of the vehicle interior (e.g., a driver's seat, a passenger seat, or the like) is an observation area. Furthermore, the vision sensor 55 and the radar 56 are disposed close to each other.

The description returns to FIG. 3. The analysis unit 61 includes the own position estimation unit 71, the sensor fusion unit 72, the recognition unit 73, the specifying unit 74, the acquisition unit 75, and the conversion unit 76, and implements or executes functions and actions of control processing described below.

Note that the internal configuration of the analysis unit 61 is not limited to the configuration illustrated in FIG. 3, and may be another configuration as long as the another configuration performs control processing to be described later. Furthermore, since the own position estimation unit 71, the sensor fusion unit 72, and the recognition unit 73 have been described above, detailed description thereof will be omitted.

First, as illustrated in FIG. 5, the specifying unit 74 (see FIG. 3) specifies the position of the chest of the passenger (e.g., driver D) in the vehicle by the vision sensor 55 (step S01). The passenger and the driver D are examples of measurement targets.

For example, the specifying unit 74 detects the position of each part of a human body (driver D) by performing known image processing on images captured by the vision sensor 55, and specifies the position of the chest from each detected part.

Furthermore, since the chest has the heart and the lung that are examples of the vibration source, the specifying unit 74 specifies the position of the chest, so that it is possible to specify the position of the vibration source in the driver D.

Next, as illustrated in FIG. 6, the acquisition unit 75 (see FIG. 3) acquires by the radar 56 vibration information of the chest whose position has been specified by the specifying unit 74 (see FIG. 3) (step S02).

More specifically, the acquisition unit 75 measures by the radar 56 fluctuation (temporal transition) of a distance to an object in the same direction as that of the position of the chest identified by the specifying unit 74, and acquires vibration information of the chest based on the fluctuation of the measured distance.

Note that, in the present disclosure, while fluctuation of heartbeat is about 0.1 to 0.5 (mm) and fluctuation of respiration is about 1 to 12 (mm), the wavelength of the millimeter wave is one (cm) or less. Consequently, the phase of the millimeter wave is shifted due to the fluctuation of the heartbeat or the respiration, so that it is possible to acquire the vibration information of the chest without any problem by acquiring the change in the phase by the radar 56.

Next, subsequently to the processing in above-described step S02, the conversion unit 76 (see FIG. 3) converts the vibration information of the chest acquired by the acquisition unit 75 into vital information (e.g., the heart rate or the respiratory rate) of the driver D (step S03).

For example, the conversion unit 76 extracts vibration having a frequency (e.g., the heart rate is about 60 to 100 (times/minute)) based on which the heart rate or the respiratory rate can be estimated, as vital information from the vibration information of the chest, and thereby converts the vibration information of the chest into the vital information of the driver D.

Here, in the embodiment, the radio wave used by the radar 56 is in a band at which the radio wave transmits through clothes, so that it is possible to directly detect movement of the skin of the driver D by the radar 56. Consequently, in the embodiment, it is possible to accurately detect movement of the chest of the driver D.

Furthermore, in the embodiment, the vision sensor 55 specifies the position of the chest of the driver D, and the vibration information of the chest is acquired based on the specified position of the chest, so that it is possible to accurately detect the movement of the chest of the driver D even in an environment of the vehicle interior including many reflection objects.

As described above, in the embodiment, the specifying unit 74 and the acquisition unit 75 interlock the vision sensor 55 and the radar 56, so that it is possible to accurately detect micro vibrations caused by the heartbeat, the respiration, or the like of the driver D.

Furthermore, in the embodiment, the vision sensor 55 and the radar 56 is preferably disposed close to each other. Consequently, it is possible to accurately interlock with the acquisition unit 75 the position information of the chest specified by the specifying unit 74, so that it is possible to more accurately detect micro vibrations caused by the heartbeat, the respiration, or the like of the driver D.

In the embodiment, the radar 56 is preferably disposed on the front side (e.g., the front side of the vehicle interior of the vehicle 1) of the measurement target (here, the driver D). Consequently, the radar 56 can measure the chest on the front side on which fluctuation is greater than that of the chest on the back side at the time of respiration or the like, so that it is possible to more accurately detect micro vibration caused by the heartbeat, respiration, or the like of the driver D.

### Modification 1

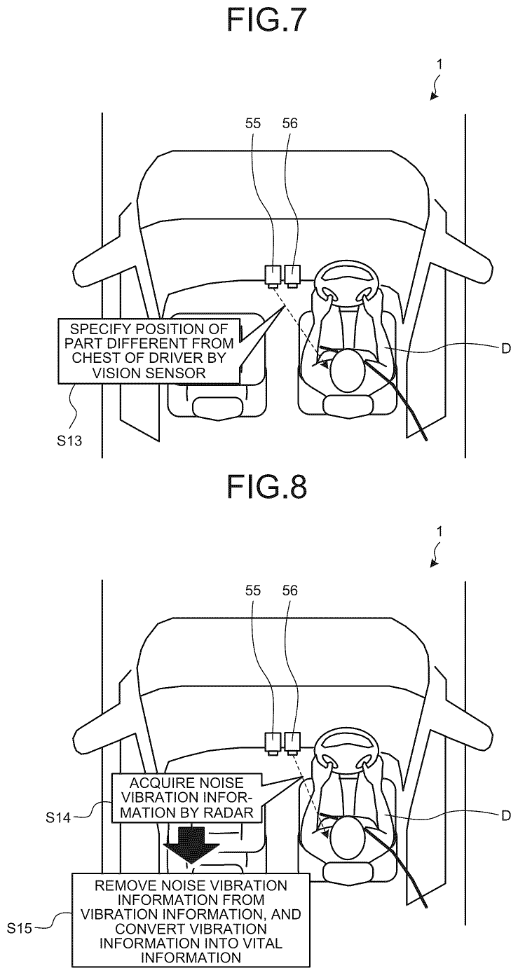

Next, details of detection processing according to various modifications of the embodiment will be described. FIGS. 7 and 8 are diagrams for describing examples of processing executed by the vehicle control system 11 according to modification 1 of the embodiment of the present disclosure.

In this modification 1, first, the specifying unit 74 (see FIG. 3) specifies the position of the chest of the driver D (see FIG. 5) in the vehicle by the vision sensor 55 (see FIG. 3) (step S11). Next, the acquisition unit 75 (see FIG. 3) acquires by the radar 56 (see FIG. 3) the vibration information of the chest whose position has been specified by the specifying unit 74 (step S12).

Note that, since the processing in steps S11 and S12 is similar to the processing in above-described steps S01 and S02, detailed description thereof will be omitted.

Next, as illustrated in FIG. 7, the specifying unit 74 (see FIG. 3) specifies a position of a part different from the chest of the driver D by the vision sensor 55 (step S13).

For example, the specifying unit 74 detects the position of each part of the driver D by performing the known image processing on images captured by the vision sensor 55, and specifies the position of a part (e.g., a shoulder, a neck, or the like) different from the chest from each detected part.

Next, as illustrated in FIG. 8, the acquisition unit 75 (see FIG. 3) acquires by the radar 56 noise vibration information that is vibration information of the part whose position has been specified by the specifying unit 74 (see FIG. 3) (step S14).

More specifically, the acquisition unit 75 measures by the radar 56 fluctuation (temporal transition) of the distance to the object in the same direction as that of the position of the part specified by the specifying unit 74, and acquires the noise vibration information based on the fluctuation of the measured distance.

Next, the conversion unit 76 (see FIG. 3) removes the noise vibration information of the part different from the chest from the vibration information of the chest acquired by the acquisition unit 75, and converts the vibration information from which the noise vibration information has been removed into vital information of the driver D (step S15).

Consequently, it is possible to acquire the vital information from which micro vibration components have been removed even in an environment such as the vehicle interior during traveling where micro vibrations constantly occur. Consequently, according to modification 1, it is possible to more accurately detect micro vibrations caused by heartbeat, respiration, or the like of the driver D.

Furthermore, in modification 1, the part for which the noise vibration information is acquired is preferably a part that is close to the chest and at which the skin is exposed. Consequently, it is possible to acquire the accurate noise vibration information, so that it is possible to more accurately detect micro vibrations caused by heartbeat, respiration, or the like of the driver D.

### Modification 2

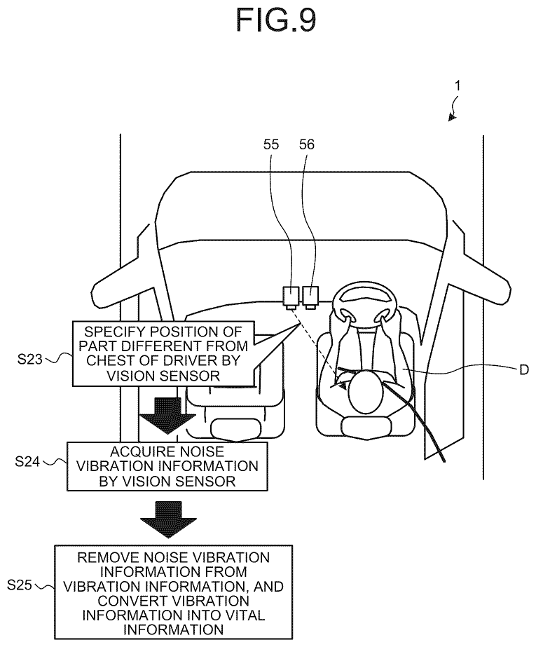

FIG. 9 is a diagram for describing an example of processing executed by the vehicle control system 11 according to modification 2 of the embodiment of the present disclosure.

In this modification 2, first, the specifying unit 74 (see FIG. 3) specifies the position of the chest of the driver D (see FIG. 5) in the vehicle by the vision sensor 55 (see FIG. 3) (step S21). Next, the acquisition unit 75 (see FIG. 3) acquires by the radar 56 (see FIG. 3) the vibration information of the chest whose position has been specified by the specifying unit 74 (step S22).

Note that, since the processing in steps S21 and S22 is similar to the processing in above-described steps S01 and S02, detailed description thereof will be omitted.

Next, as illustrated in FIG. 9, the specifying unit 74 (see FIG. 3) specifies a position of a part different from the chest of the driver D by the vision sensor 55 (step S23).

For example, the specifying unit 74 detects the position of each part of the driver D by performing the known image processing on images captured by the vision sensor 55, and specifies the position of the part (e.g., the shoulder, the neck, or the like) different from the chest from each detected part.

Next, the acquisition unit 75 (see FIG. 3) acquires by the vision sensor 55 the noise vibration information that is vibration information of the part whose position has been specified by the specifying unit 74 (step S24).

More specifically, the acquisition unit 75 measures by the vision sensor 55 fluctuation (temporal transition) of the position of the part specified by the specifying unit 74, and acquires the noise vibration information based on the measured fluctuation.

Next, the conversion unit 76 (see FIG. 3) removes the noise vibration information of the part different from the chest from the vibration information of the chest acquired by the acquisition unit 75, and converts the vibration information from which the noise vibration information has been removed into vital information of the driver D (step S25).

Consequently, it is possible to acquire the vital information from which micro vibration components have been removed even in an environment such as the vehicle interior during traveling where micro vibrations constantly occur. Consequently, according to modification 2, it is possible to more accurately detect micro vibrations caused by heartbeat, respiration, or the like of the driver D.

Furthermore, in modification 2, the part for which the noise vibration information is acquired is preferably a part that is close to the chest and at which the skin is exposed. Consequently, it is possible to acquire the accurate noise vibration information, so that it is possible to more accurately detect micro vibrations caused by heartbeat, respiration, or the like of the driver D.

Note that, in modification 1 and modification 2, the part for which the noise vibration information is acquired may be a part distant from the chest or a part at which the skin is not exposed. Furthermore, in modification 1 and modification 2, vibration at portions other than the driver D may be acquired as the noise vibration information.

### Modification 3

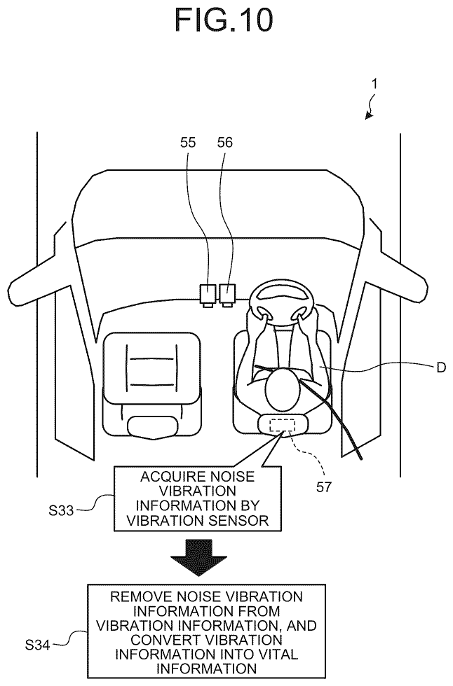

FIG. 10 is a diagram for describing an example of processing executed by the vehicle control system 11 according to modification 3 of the embodiment of the present disclosure.

In this modification 3, first, the specifying unit 74 (see FIG. 3) specifies the position of the chest of the driver D (see FIG. 5) in the vehicle by the vision sensor 55 (see FIG. 3) (step S31). Next, the acquisition unit 75 (see FIG. 3) acquires by the radar 56 (see FIG. 3) the vibration information of the chest whose position has been specified by the specifying unit 74 (step S32).

Note that, since the processing in steps S31 and S32 is similar to the processing in above-described steps S01 and S02, detailed description thereof will be omitted.

Next, the acquisition unit 75 acquires noise vibration information by a vibration sensor 57 installed in the vehicle interior of the vehicle 1 (step S33). For example, the acquisition unit 75 acquires as noise vibration information the vibration information detected by the vibration sensor 57 installed in a driver's seat or the like.

Next, the conversion unit 76 removes the noise vibration information detected by the vibration sensor 57 from the vibration information of the chest acquired by the acquisition unit 75, and converts the vibration information from which the noise vibration information has been removed into the vital information of the driver D (step S34).

Consequently, it is possible to acquire the vital information from which micro vibration components have been removed even in an environment such as the vehicle interior during traveling where micro vibrations constantly occur. Consequently, according to modification 3, it is possible to more accurately detect micro vibrations caused by heartbeat, respiration, or the like of the driver D.

Furthermore, in modification 3, a portion at which the vibration sensor 57 is installed is preferably near (at, for example, the driver's seat or the like of) the measurement target (here, the driver D). Consequently, it is possible to acquire the accurate noise vibration information, so that it is possible to more accurately detect micro vibrations caused by heartbeat, respiration, or the like of the driver D.

Note that, in modification 3, the portion at which the vibration sensor 57 is installed may be a portion distant from the driver's seat in the vehicle 1.

### Modification 4

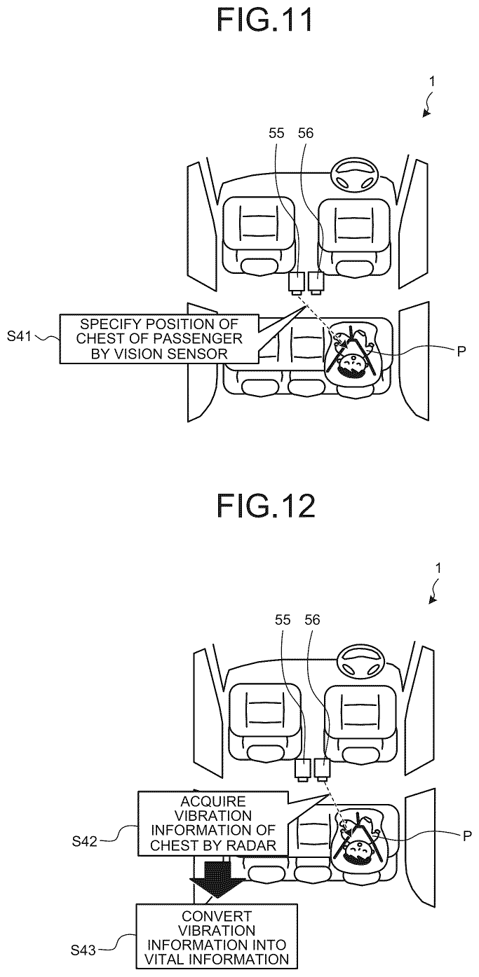

Although the embodiment and the various modifications described so far have described the examples where the vital information of the driver D is acquired, the present disclosure is not limited to these examples. FIGS. 11 and 12 are diagrams for describing an example of processing executed by the vehicle control system 11 according to modification 4 of the embodiment of the present disclosure.

As illustrated in FIG. 11, in modification 4, the vision sensor 55 and the radar 56 are disposed at the center part (e.g., between the driver's seat and the rear seat, and the like) of the vehicle 1, and are installed such that a predetermined area (e.g., a rear seat or the like) of the vehicle interior becomes an observation area. Furthermore, the vision sensor 55 and the radar 56 are disposed close to each other.

Furthermore, the specifying unit 74 (see FIG. 3) specifies a position of the chest of a passenger P in the vehicle interior by the vision sensor 55 (step S41). The passenger P is another example of the measurement target.

For example, the specifying unit 74 detects the position of each part of a human body (passenger P) by performing the known image processing on images captured by the vision sensor 55, and specifies the position of the chest from each detected part.

Next, as illustrated in FIG. 12, the acquisition unit 75 (see FIG. 3) acquires by the radar 56 vibration information of the chest whose position has been specified by the specifying unit 74 (see FIG. 3) (step S42).

More specifically, the acquisition unit 75 measures by the radar 56 fluctuation (temporal transition) of a distance to an object in the same direction as that of the position of the chest identified by the specifying unit 74, and acquires vibration information of the chest based on the fluctuation of the measured distance.

Next, the conversion unit 76 (see FIG. 3) converts the vibration information of the chest acquired by the acquisition unit 75 into vital information (e.g., the heart rate, the respiratory rate, and the like) of the passenger P (step S43).

As described above, in modification 4, it is also possible to accurately detect micro vibrations caused by heartbeat, respiration, or the like for the passenger P on the rear seat. Consequently, according to modification 4, it is possible to accurately detect, for example, a child or the like left in the vehicle compartment.

Furthermore, in modification 4, the radar 56 is preferably disposed on the front side (e.g., in front of the rear seat in the vehicle 1,) of the measurement target (here, the passenger P). Consequently, the radar 56 can measure the chest on the front side on which fluctuation is greater than that of the chest on the back side at the time of respiration or the like, so that it is possible to accurately detect micro vibrations caused by heartbeat, respiration, or the like of the passenger P.

Furthermore, this modification 4 is applicable not only to the passenger P on the rear seat, but also to the passenger on the passenger seat.

Note that, although the embodiment and the various modifications described so far has described the examples where the vital information of the passenger of the vehicle 1 is acquired, the present disclosure is not limited to these examples, and the vital information of the human body that exists in a place different from the inside of the vehicle 1 may be acquired.

Furthermore, although the above-described embodiment and various modifications have described examples where a human body is a detection target, the present disclosure is not limited to these examples, and an organism other than people, an inorganic object (e.g., a machine or the like) having a vibration source inside, or the like may be a detection target.

### Procedure of Detection Processing

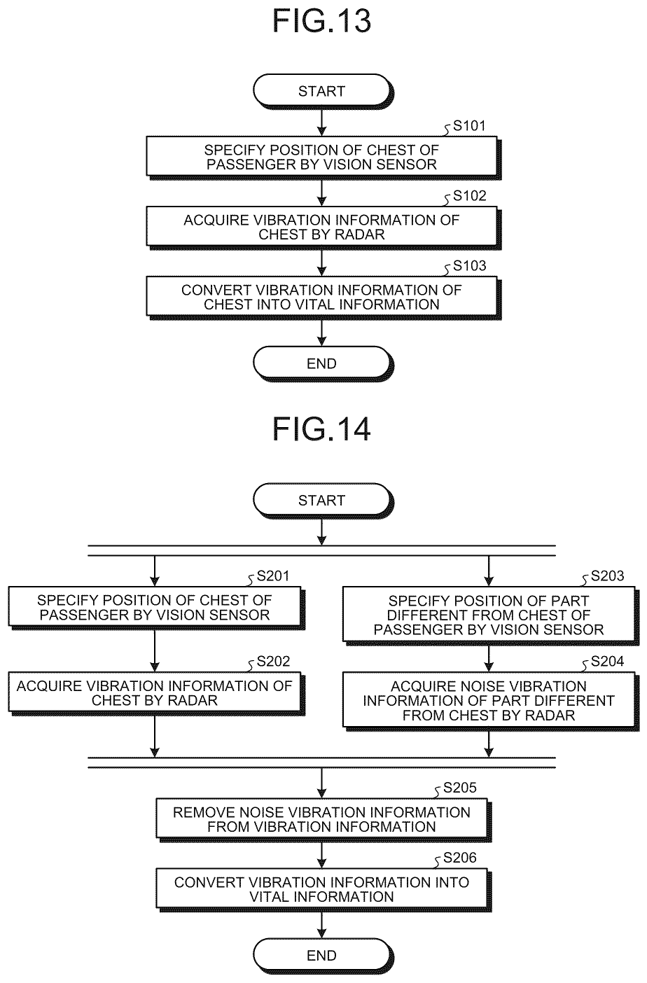

Next, a procedure of detection processing according to the embodiment and the various modifications will be described with reference to FIGS. 13 to 16. FIG. 13 is a flowchart illustrating an example of the procedure of the detection processing executed by the vehicle control system 11 according to the embodiment of the present disclosure.

First, the traveling assistance/automatic driving control unit 29 specifies the position of the chest of the passenger by the vision sensor 55 (step S101).

Furthermore, the traveling assistance/automatic driving control unit 29 acquires by the radar 56 the vibration information of the chest whose position has been specified (step S102).

Next, the traveling assistance/automatic driving control unit 29 converts the acquired vibration information of the chest into vital information of the passenger (step S103), and finishes the processing.

FIG. 14 is a flowchart illustrating an example of the procedure of the detection processing executed by the vehicle control system 11 according to modification 1 of the embodiment of the present disclosure.

First, the traveling assistance/automatic driving control unit 29 specifies the position of the chest of the passenger by the vision sensor 55 (step S201). Furthermore, the traveling assistance/automatic driving control unit 29 acquires by the radar 56 vibration information of the chest whose position has been specified (step S202).

Furthermore, in parallel with the processing in steps S201 and S202, the traveling assistance/automatic driving control unit 29 specifies the position of a part different from the chest of the passenger by the vision sensor 55 (step S203). Furthermore, the traveling assistance/automatic driving control unit 29 acquires by the radar 56 noise vibration information that is the vibration information of the part different from the chest (step S204).

Next, the traveling assistance/automatic driving control unit 29 removes the noise vibration information from the vibration information of the chest (step S205). Furthermore, the traveling assistance/automatic driving control unit 29 converts the vibration information from which the noise vibration information has been removed into vital information of the passenger (step S206), and finishes the processing.

Note that, although the example in FIG. 14 has described the case where the processing in steps S201 and S202 and the processing in steps S203 and S204 are performed in parallel, the present disclosure is not limited to these examples, and one processing group may be performed prior to an other processing group.

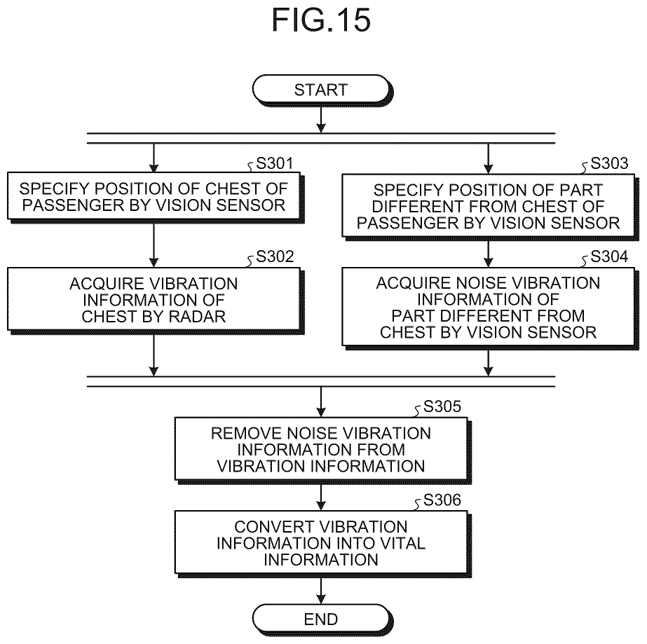

FIG. 15 is a flowchart illustrating an example of the procedure of the detection processing executed by the vehicle control system 11 according to modification 2 of the embodiment of the present disclosure.

First, the traveling assistance/automatic driving control unit 29 specifies the position of the chest of the passenger by the vision sensor 55 (step S301). Furthermore, the traveling assistance/automatic driving control unit 29 acquires by the radar 56 vibration information of the chest whose position has been specified (step S302).

Furthermore, in parallel with the processing in steps S301 and S302, the traveling assistance/automatic driving control unit 29 specifies the position of a part different from the chest of the passenger by the vision sensor 55 (step S303). Furthermore, the traveling assistance/automatic driving control unit 29 acquires by the vision sensor 55 noise vibration information that is the vibration information of the part different from the chest (step S304).

Next, the traveling assistance/automatic driving control unit 29 removes the noise vibration information from the vibration information of the chest (step S305). Furthermore, the traveling assistance/automatic driving control unit 29 converts the vibration information from which the noise vibration information has been removed into vital information of the passenger (step S306), and finishes the processing.

Note that, although the example in FIG. 15 has described the case where the processing in steps S301 and S302 and the processing in steps S303 and S304 are performed in parallel, the present disclosure is not limited to these examples, and one processing group may be performed prior to an other processing group.

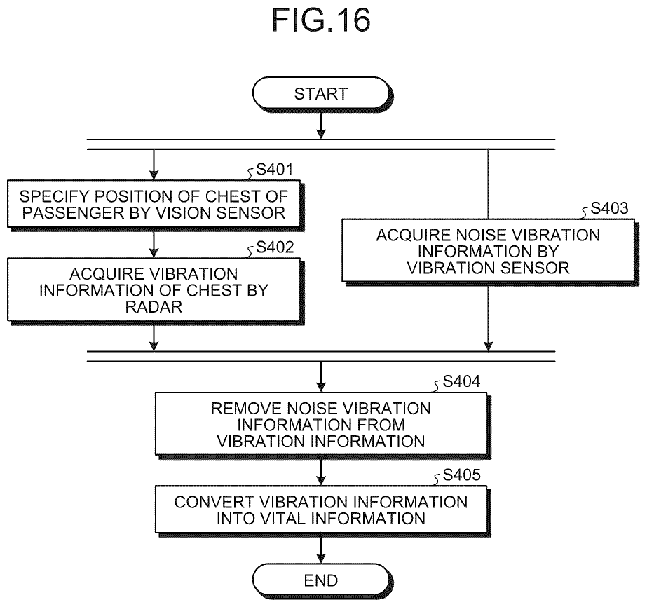

FIG. 16 is a flowchart illustrating an example of the procedure of the detection processing executed by the vehicle control system 11 according to modification 3 of the embodiment of the present disclosure.

First, the traveling assistance/automatic driving control unit 29 specifies the position of the chest of the passenger by the vision sensor 55 (step S401). Furthermore, the traveling assistance/automatic driving control unit 29 acquires by the radar 56 vibration information of the chest whose position has been specified (step S402).

Furthermore, in parallel with the processing in steps S401 and S402, the traveling assistance/automatic driving control unit 29 acquires noise vibration information by the vibration sensor 57 (step S403).

Next, the traveling assistance/automatic driving control unit 29 removes the noise vibration information from the vibration information of the chest (step S404). Furthermore, the traveling assistance/automatic driving control unit 29 converts the vibration information from which the noise vibration information has been removed into vital information of the passenger (step S405), and finishes the processing.

Note that, although the example in FIG. 16 has described the case where the processing in steps S401 and S402 and the processing in step S403 is performed in parallel, the present disclosure is not limited to these examples, and one processing group may be performed prior to an other processing group.

### Effects

A vibration detection system (vehicle control system 11) according to the embodiment includes the vision sensor 55, the radar 56, and a control unit (traveling assistance/automatic driving control unit 29) that controls the vision sensor 55 and the radar 56. Furthermore, the control unit (traveling assistance/automatic driving control unit 29) includes the specifying unit 74 and the acquisition unit 75. The specifying unit 74 specifies the position of the vibration source in the measurement target by the vision sensor 55. The acquisition unit 75 acquires vibration information related to vibration at the specified position of the vibration source by the radar 56.

Consequently, it is possible to accurately detect micro vibrations caused by heartbeat, respiration, or the like of the passenger.

Furthermore, in the vibration detection system (vehicle control system 11) according to the embodiment, the vision sensor 55 and the radar 56 are disposed close to each other.

Consequently, it is possible to more accurately detect micro vibrations caused by the heartbeat, the respiration, or the like of the passenger.

Furthermore, in the vibration detection system (vehicle control system 11) according to the embodiment, the acquisition unit 75 removes, from the vibration information, noise vibration information related to vibration at a position different from the position of the vibration source in the measurement target.

Consequently, it is possible to more accurately detect micro vibrations caused by the heartbeat, the respiration, or the like of the passenger.

Furthermore, in the vibration detection system (vehicle control system 11) according to the embodiment, the noise vibration information is acquired by the radar 56.

Consequently, it is possible to more accurately detect micro vibrations caused by the heartbeat, the respiration, or the like of the passenger.

Furthermore, in the vibration detection system (vehicle control system 11) according to the embodiment, the noise vibration information is acquired by the vision sensor 55.

Consequently, it is possible to more accurately detect micro vibrations caused by the heartbeat, the respiration, or the like of the passenger.

Furthermore, in the vibration detection system (vehicle control system 11) according to the embodiment, the acquisition unit 75 removes the noise vibration information acquired by the vibration sensor 57 from the vibration information.

Consequently, it is possible to more accurately detect micro vibrations caused by the heartbeat, the respiration, or the like of the passenger.

Furthermore, in the vibration detection system (vehicle control system 11) according to the embodiment, the vision sensor 55 and the radar 56 are installed such that the inside of the vehicle 1 is an observation area.

Consequently, it is possible to accurately detect micro vibrations caused by the heartbeat, the respiration, or the like of the passenger.

Furthermore, in the vibration detection system (vehicle control system 11) according to the embodiment, the measurement target is a human body, and the vibration source is at least one of the heart and the lung.

Consequently, it is possible to accurately detect vital information of the human body.

Furthermore, the vibration detection method according to the embodiment is a vibration detection method executed by a computer, and includes a specifying step (steps S101, S201, S301, and S401) and an acquisition step (steps S102, S202, S302, and S402). In the specifying step (steps S101, S201, S301, and S401), the position of the vibration source in the measurement target is specified by the vision sensor 55. In the acquisition step (steps S102, S202, S302, and S402), the vibration information related to vibration at the specified position of the vibration source is acquired by the radar 56.

Consequently, it is possible to accurately detect micro vibrations caused by the heartbeat, the respiration, or the like of the passenger.

Although the embodiment of the present disclosure has been described above, the technical scope of the present disclosure is not limited to the above-described embodiment as is, and various modifications can be made without departing from the gist of the present disclosure. Furthermore, components according to different embodiments and modifications may be appropriately combined.

Furthermore, the effects described in the description are merely examples and are not limited, and other effects may be provided.

Note that the technique according to the present disclosure can also employ the following configurations.

1

A vibration detection system comprising:
a vision sensor;
a radar; and
a control unit that controls the vision sensor and the radar, wherein
the control unit includes
a specifying unit that specifies a position of a vibration source in a measurement target by the vision sensor, and
an acquisition unit that acquires vibration information related to vibration at the specified position of the vibration source by the radar.
   2
The vibration detection system according to the above (1), wherein
the vision sensor and the radar are disposed close to each other.
   3

The vibration detection system according to the above (1) or (2), wherein
the acquisition unit removes noise vibration information from the vibration information, the noise vibration information being related to vibration at a position different from the position of the vibration source in the measurement target.
   4

The vibration detection system according to the above (3), wherein
the noise vibration information is acquired by the radar.
   5

The vibration detection system according to the above (3) or (4), wherein
the noise vibration information is acquired by the vision sensor.
   6

The vibration detection system according to any one of the above (1) to (5), wherein
the acquisition unit removes, from the vibration information, noise vibration information acquired by a vibration sensor.
   7

The vibration detection system according to any one of the above (1) to (6), wherein
the vision sensor and the radar are installed such that an inside of a vehicle is an observation area.
   8

The vibration detection system according to any one of the above (1) to (7), wherein
the measurement target is a human body, and
the vibration source is at least one of a heart and a lung.
   9

A vibration detection method executed by a computer comprising:
a specifying step of specifying a position of a vibration source in a measurement target by a vision sensor; and
an acquisition step of acquiring vibration information related to vibration at the specified position of the vibration source by a radar.
   10

The vibration detection method according to the above (9), wherein
the vision sensor and the radar are disposed close to each other.
   11

The vibration detection method according to the above (9) or (10), wherein
the acquisition step removes noise vibration information from the vibration information, the noise vibration information being related to vibration at a position different from the position of the vibration source in the measurement target.
   12

The vibration detection method according to the above (11), wherein
the noise vibration information is acquired by the radar.
   13

The vibration detection method according to the above (11) or (12), wherein
the noise vibration information is acquired by the vision sensor.
   14

The vibration detection method according to any one of the above (9) to (13), wherein
the acquisition step removes, from the vibration information, noise vibration information acquired by a vibration sensor.
   15

The vibration detection method according to any one of the above (9) to (14), wherein
the vision sensor and the radar are installed such that an inside of a vehicle is an observation area.
   16

The vibration detection method according to any one of the above (9) to (15), wherein
the measurement target is a human body, and
the vibration source is at least one of a heart and a lung.

### Reference Signs List

1 VEHICLE
26 INTRA-VEHICLE SENSOR
29 TRAVELING ASSISTANCE/AUTOMATIC DRIVING CONTROL UNIT (EXAMPLE OF CONTROL UNIT)
55 VISION SENSOR
56 RADAR
61 ANALYSIS UNIT
74 SPECIFYING UNIT
75 ACQUISITION UNIT
76 CONVERSION UNIT
D DRIVER (EXAMPLE OF MEASUREMENT TARGET)
P PASSENGER (EXAMPLE OF MEASUREMENT TARGET)

1. A vibration detection system comprising:
a vision sensor;
a radar; and
a control unit that controls the vision sensor and the radar, wherein
the control unit includes
a specifying unit that specifies a position of a vibration source in a measurement target by the vision sensor, and
an acquisition unit that acquires vibration information related to vibration at the specified position of the vibration source by the radar.

2. The vibration detection system according to claim 1, wherein
the vision sensor and the radar are disposed close to each other.

3. The vibration detection system according to claim 1, wherein
the acquisition unit removes noise vibration information from the vibration information, the noise vibration information being related to vibration at a position different from the position of the vibration source in the measurement target.

4. The vibration detection system according to claim 3, wherein
the noise vibration information is acquired by the radar.

5. The vibration detection system according to claim 3, wherein
the noise vibration information is acquired by the vision sensor.

6. The vibration detection system according to claim 1, wherein
the acquisition unit removes, from the vibration information, noise vibration information acquired by a vibration sensor.

7. The vibration detection system according to claim 1, wherein
the vision sensor and the radar are installed such that an inside of a vehicle is an observation area.

8. The vibration detection system according to claim 1, wherein
the measurement target is a human body, and
the vibration source is at least one of a heart and a lung.

9. A vibration detection method executed by a computer comprising:
a specifying step of specifying a position of a vibration source in a measurement target by a vision sensor; and
an acquisition step of acquiring vibration information related to vibration at the specified position of the vibration source by a radar.
